(19) United States
(12) Patent Application Publication
(10) Pub. No.: US 2009/0086885 A1
(43) Pub. Date: Apr. 2, 2009

(54) RADIATION IMAGE CAPTURING APPARATUS AND METHOD

(75) Inventors: Yoshitaka YAMAGUCHI, Minami-ashigara-shi (JP); Sadato Akahori, Odawara-shi (JP); Kazuharu Ueta, Tokyo (JP); Yasunori Ohta, Yokohama-shi (JP); Atsushi Fukuda, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/239,395

(22) Filed: Sep. 26, 2008

(30) Foreign Application Priority Data
Sep. 27, 2007 (JP) 2007-251760

(51) Int. Cl. A61B 6/00

(52) U.S. Cl. 378/8

(57) ABSTRACT

A radiation image capturing apparatus for producing radiation image information of a subject by applying a radiation to the subject on which a fixed member is mounted, including a subject image information acquiring unit for acquiring subject image information of the subject including the fixed member, an imaging region identifier for processing the acquired subject image information to identify an imaging region of the subject with the fixed member mounted thereon, an irradiated region controller for controlling an irradiated region to be irradiated with the radiation in order to apply the radiation to the identified imaging region, and a radiation conversion panel for converting the radiation that has passed through the imaging region into radiation image information.

### BACKGROUND OF THE INVENTION

1. Field of the Invention:

The present invention relates to a radiation image capturing apparatus and method for producing radiation image information of a subject by applying a radiation to the subject on which a fixed member such as a plaster cast, glass fibers, etc. is mounted.

2. Description of the Related Art:

In the medical field, there have widely been used radiation image capturing apparatus which apply a radiation from a radiation source to a subject (a patient) and guide the radiation that has passed through the subject to a radiation conversion panel, which converts the radiation into radiation image information, after which the radiation image information is processed. The processed radiation image information is displayed by a display device for diagnostic purposes.

Existing radiation conversion panels include a solid-state detector for converting the radiation into electric charge information, storing the electric charge information, and reading the stored electric charge information as an electric signal, and a stimulable phosphor panel for storing radiation energy in a phosphor and emitting stimulated light depending on the stored radiation energy when irradiated with stimulating light such as a laser beam or the like.

It has been put to practical use a process of extracting image information from a soft tissue which is a region of interest (ROI) of the subject, such as a heart, a lung, etc. disposed below ribs, for example, using the radiation image capturing apparatus. According to the process, because a bone such as a rib and a soft tissue such as a heart have different radiation absorbing characteristics, radiations having different energy levels are applied to the subject to acquire two types of radiation image information under different image capturing conditions, and the difference between the two types of radiation image information is determined by certain weighting, thereby extracting image information of the bone or the soft tissue (see Japanese Laid-Open Patent Publication No. 2002-325756).

If the region of interest of the subject is a bone fracture region and a plaster cast is mounted as a fixed member on the bone fracture region, then since the plaster cast has considerably larger radiation absorbing characteristics than the other regions not covered with the plaster cast, the dosage of a radiation to be applied to the subject has to be greater than usual in order to obtain desired radiation image information of the bone fracture region. Unless the radiation is applied exactly to the region of interest through the plaster cast, the other regions of the subject which are free of the plaster cast tend to be irradiated with the greater dosage of the radiation than usual.

For applying a radiation highly accurately to a desired position on a subject, Japanese Laid-Open Patent Publication No. 2004-283367 discloses a technology to acquire an optical image from within a region of the subject which is to be irradiated with a radiation and adjust the position of the radiation source or the subject based on the optical image in order to bring the region of the subject into an appropriate position.

According to Japanese Laid-Open Patent Publication No. 2004-283367, however, only the region of the subject which is to be irradiated with the radiation is confirmed and adjusted in position. There is nothing in the publication which shows a process of identifying a region in which a plaster cast is mounted on the subject and adjust the region of the subject which is to be irradiated with the radiation depending on the identified region.

### SUMMARY OF THE INVENTION

It is a general object of the present invention to provide a radiation image capturing apparatus and method for producing radiation image information of a subject without the need for applying an excessive amount of a radiation to the subject by setting highly accurately a region of the subject which is to be irradiated with the radiation depending on an imaging region of the subject with a fixed member mounted thereon.

The above and other objects, features, and advantages of the present invention will become more apparent from the following description when taken in conjunction with the accompanying drawings in which a preferred embodiment of the present invention is shown by way of illustrative example.

### BRIEF DESCRIPTION OF THE DRAWINGS

FIG. 1 is a block diagram of a radiation image capturing apparatus according to an embodiment of the present invention;

FIG. 2 is a side elevational view of the radiation image capturing apparatus;

FIG. 3 is a block diagram of a radiation solid-state detecting device of the radiation image capturing apparatus; and

FIG. 4 is a flowchart of an operation sequence of the radiation image capturing apparatus.

### DESCRIPTION OF THE PREFERRED EMBODIMENT

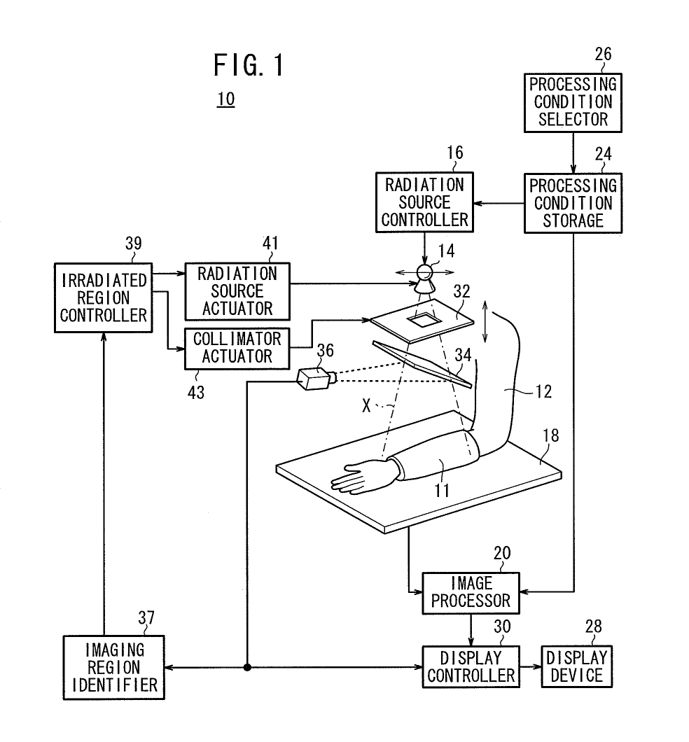

FIG. 1 shows in block form a radiation image capturing apparatus 10 according to an embodiment of the present invention.

As shown in FIG. 1, the radiation image capturing apparatus 10 comprises a radiation source 14 for applying a radiation X to a subject 12 with a fixed member 11 such as a plaster cast or glass fibers mounted thereon, a radiation source controller 16 for controlling the radiation source 14 under image capturing conditions including a tube voltage, a tube current, an irradiation time, etc. that have been set, a radiation solid-state detector (radiation conversion panel) 18 for converting the radiation X that has passed through the subject 12 into radiation image information as electric charge information, an image processor 20 for processing the radiation image information detected by the radiation solid-state detector 18, a processing condition storage 24 for storing processing conditions including the image capturing conditions, a processing condition selector 26 for selecting processing conditions for obtaining desired radiation image information from the processing condition storage 24, a display device 28 for displaying the radiation image information processed by the image processor 20, and a display controller 30 for controlling the display device 28.

The image processor 20 determines radiation image information of a desired region to be imaged of the subject by performing a weighted subtractive process on a plurality of pieces of radiation image information that are generated when radiations X having different energy levels are applied to the subject 12 with the fixed member 11 mounted thereon. The weighted subtractive process is a process of determining processed radiation image information S according to the following equation:

S=α·S₁+S₂

where S₁ represents radiation image information obtained under a first image capturing condition, S₂ represents radiation image information obtained under a second image capturing condition, α a weighting coefficient.

In order to optimize the contrast and density of an image of the desired region to be imaged from which the image of the fixed member 11 is removed, the processed radiation image information S may be determined according to the following equation:

S=K₁·S₁+K₂·S₂+K₃

where K₁, K₂, K₃ represent coefficients that are determined by the weighting coefficient for extracting the region to be imaged by removing the image of the fixed member 11 and the gradation characteristics of the image of the region to be imaged.

The first image capturing condition, the second image capturing condition, and the weighting coefficient α are established depending on the type of the fixed member 11 mounted on the subject 12 and the region to be imaged, as well as taking into account the minimization of the dosage of the radiation applied to the subject 12. The first image capturing condition and the second image capturing condition are conditions concerning the tube voltage and the tube current that are set in the radiation source 14.

The processing conditions may include the first image capturing condition and the second image capturing condition that are fixed and the weighting coefficient α that is established depending on the type of the fixed member 11 and the region to be imaged. Alternatively, the processing conditions may include the weighting coefficient α that is fixed and the first image capturing condition and the second image capturing condition that are established depending on the type of the fixed member 11 and the region to be imaged. If the fixed member 11 is a plaster cast, then its radiation absorbing characteristics differ when its water content is high at the time the fixed member 11 is initially mounted on the subject 12 and when its water content is low at the time the fixed member 11 is solidified upon elapse of a certain period of time. Accordingly, the weighting coefficient α may be set as a function α(t) of the time t that has elapsed from the time when the fixed member 11 was mounted on the subject 12.

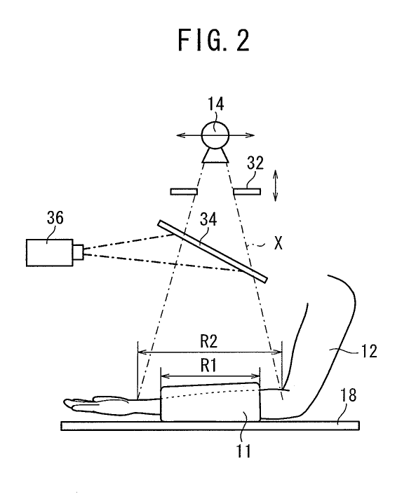

As shown in FIG. 2, the radiation image capturing apparatus 10 includes a collimator 32 disposed between the radiation source 14 and the radiation solid-state detector 18 for adjusting a region of the subject 12 that is to be irradiated with the radiation X (irradiated region), a half-silvered mirror 34 disposed between the collimator 32 and the radiation solid-state detector 18, and a CCD camera 36 for capturing an image (subject image information) of the subject 12, which includes the fixed member 11 disposed in a given position on the radiation solid-state detector 18, via the half-silvered mirror 34. The subject image information acquired by the CCD camera 36 is supplied to an imaging region identifier 37 (FIG. 1) which processes the subject image information to calculate a region R1 (imaging region) of the fixed member 11 and a present irradiated region R2 of the subject 12 that is irradiated with the radiation X. The imaging region identifier 37 supplies the information of the region R1 and the irradiated region R2 to an irradiated region controller 39. The irradiated region controller 39 controls a radiation source actuator (irradiated region moving means) 41 for moving the radiation source 14 along the plane of the radiation solid-state detector 18 and a collimator actuator (irradiated region adjusting means) 43 for moving the collimator 32 along the major axis of the radiation X, based on the information of the region R1 and the irradiated region R2 supplied from the imaging region identifier 37.

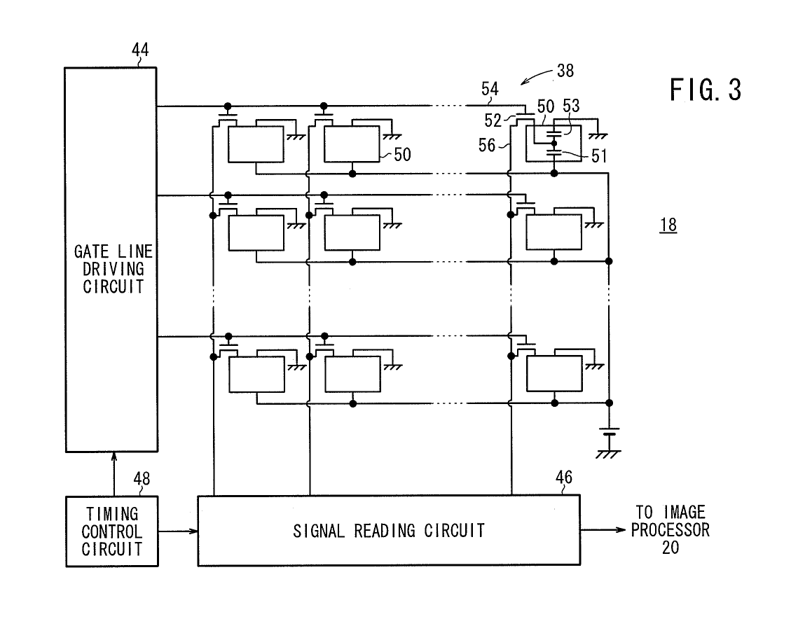

FIG. 3 shows the radiation solid-state detector 18 in block form. As shown in FIG. 3, the radiation solid-state detector 18 comprises a sensor substrate 38, a gate line driving circuit 44, a signal reading circuit 46, and a timing control circuit 48 for controlling the gate line driving circuit 44 and the signal reading circuit 46.

The sensor substrate 38 comprises an array of thin-film transistors (TFTs) 52 arranged in rows and columns, a photoelectric conversion layer 51 made of a material such as amorphous selenium (a-Se) for generating electric charges upon detection of the radiation X, the photoelectric conversion layer 51 being disposed on the array of TFTs 52, and an array of storage capacitors 53 connected to the photoelectric conversion layer 51. When the radiation X is applied to the sensor substrate 38, the photoelectric conversion layer 51 generates electric charges, and the storage capacitors 53 store the generated electric charges. Then, the TFTs 52 are turned on along each row at a time to read the electric charges from the storage capacitors 53 as an image signal. In FIG. 3, the photoelectric conversion layer 51 and one of the storage capacitors 53 are shown as a pixel 50, and the pixel 50 is connected to one of the TFTs 52. Details of the other pixels 50 are omitted from illustration. Since amorphous selenium tends to change its structure and lose its function at high temperatures, it needs to be used in a certain temperature range. The TFTs 52 connected to the respective pixels 50 are connected to respective gate lines 54 extending parallel to the rows and respective signal lines 56 extending parallel to the columns. The gate lines 54 are connected to the gate line driving circuit 44, and the signal lines 56 are connected to the signal reading circuit 46.

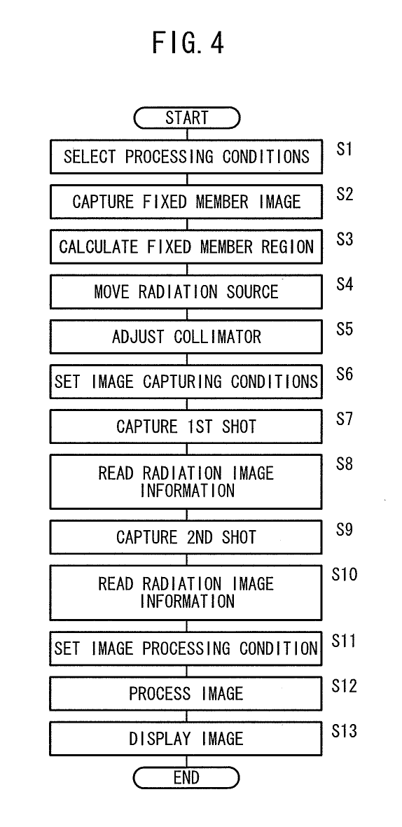

The radiation image capturing apparatus 10 is basically constructed as described above, and operation of the radiation image capturing apparatus 10 will be described below with reference to a flowchart shown in FIG. 4.

First, the operator selects desired processing conditions from the processing condition storage 24, using the processing condition selector 26 in step Si. For example, if the operator is to acquire radiation image information of an arm of the subject 12 on which the fixed member 11 in the form of a plaster cast is mounted, then the operator selects a first image capturing condition, a second image capturing condition, and a weighting coefficient α corresponding to the radiation absorbing characteristics of the plaster cast and the arm.

Then, in order to adjust the irradiated region of the subject 12 to be irradiated with the radiation X, the CCD camera 36 is energized to capture an image of the subject 12 including the fixed member 11 via the half-silvered mirror 34 in step S2. The acquired subject image information including the fixed member 11 is supplied to the imaging region identifier 37, which calculates a region R1 of the fixed member 11 included in the subject image information and a present irradiated region R2 of the subject 12 that is irradiated with the radiation X in step S3.

As shown in FIG. 2, it is assumed that the present irradiated region R2 includes a portion of the subject 12 which is not covered with the fixed member 11. The imaging region identifier 37 can calculate the region R1 of the fixed member 11 and the present irradiated region R2, from the luminance value or density value (inherent image information) of the acquired subject image information. If the fixed member 11 comprises a plaster cast which is nearly white, then its luminance value is considered to be higher than the luminance value of the subject 12 or its density value is considered to be lower than the density value of the subject 12. Accordingly, the imaging region identifier 37 can calculate the region R1 and the irradiated region R2 based on the difference between the luminance values or the density values.

The information of the region R1 and the irradiated region R2 that have been calculated is supplied to the irradiated region controller 39. Based on the information of the region R1 and the irradiated region R2, the irradiated region controller 39 controls the radiation source actuator 41 to move the radiation source 14 in step S4 and controls the collimator actuator 43 to adjust the position of the collimator 32 in step S5.

Specifically, the collimator actuator 43 moves the collimator 32 in one of the directions indicated by the arrows in FIG. 2 to adjust the distance between the radiation source 14 and the collimator 32 for thereby essentially equalizing the irradiated region R2 to the region R1. The radiation source actuator 41 moves the radiation source 14 together with the collimator 32 along the radiation solid-state detector 18 in one of the directions indicated by the arrows in FIG. 2 for thereby positionally aligning the region R1 and the irradiated region R2 with each other. As a result, the radiation X emitted from the radiation source 14 is adjusted to be applied only to the region R1 of the fixed member 11.

After the radiation source 14 and the collimator 32 are positionally adjusted, the first image capturing condition and the second image capturing condition selected from the processing condition storage 24 are set in the radiation source controller 16 in step S6.

The radiation source controller 16 with the image capturing conditions thus set therein controls the radiation source 14 under the tube voltage and the tube current based on the first image capturing condition to apply the radiation X through the fixed member 11 to the subject 12, thereby capturing a first shot in step S7. Since the irradiated region R2 has been adjusted in alignment with the region R1 of the fixed member 11 in steps S4, S5, the portion of the subject 12 which is free of the fixed member 11 is not excessively irradiated with the radiation X.

The radiation X that has passed through the fixed member 11 and the subject 12 is converted into electric signals by the photoelectric conversion layer 51 of the pixels 50 of the sensor substrate 38 of the radiation solid-state detector 18. The electric signals are stored as electric charges in the storage capacitors 53. The stored electric charges, which represent radiation image information S₁ of the first shot of the subject 12, are read from the sensor substrate 38 according to the timing control signal which is supplied from the timing control circuit 48 to the gate line driving circuit 44 and the signal reading circuit 46.

Specifically, the gate line driving circuit 44 selects one of the gate lines 54 according to the timing control signal from the timing control circuit 48, and supplies a drive signal to the bases of the TFTs 52 connected to the selected gate line 54. The signal reading circuit 46 successively switches between the signal lines 56 connected to the TFTs 52 to select one of the signal lines 56 at a time. The electric charge information representing the radiation image information S₁ that is stored in the storage capacitor 53 of the pixel 50 which corresponds to the selected gate line 54 and the selected signal line 56 is supplied as an image signal to the image processor 20. After all the image signals are read from the pixels 50 connected to the selected gate line 54, the gate line driving circuit 44 selects the next gate line 54 and supplies a drive signal to the selected gate line 54. The signal reading circuit 46 then successively reads image signals from the TFTs 52 connected to the selected gate line 54 in the same manner as described above. The above operation is repeated to read two-dimensional radiation image information S₁ stored in the sensor substrate 38, and supply the read two-dimensional radiation image information S₁ to the image processor 20 in step S8.

Then, the radiation source controller 16 controls the radiation source 14 under the tube voltage and the tube current based on the second image capturing condition to apply the radiation X through the fixed member 11 to the subject 12, thereby capturing a second shot in step S9. The second shot is captured immediately after the first shot is captured. Therefore, there is no motion artifact generated due to the motion of the subject 12 between the first shot and the second shot.

Radiation image information S₂ of the second shot which is detected by the radiation solid-state detector 18 is read and supplied to the image processor 20 in the same manner as the radiation image information Si of the first shot in step S10.

The image processor 20 has set therein the weighting coefficient α, which is one of the processing conditions selected from the processing condition storage 24 by the processing condition selector 26, in step S11.

Using the radiation image information S₁, S₂ supplied from the radiation solid-state detector 18 and the weighting coefficient α selected from the processing condition storage 24, the image processor 20 calculates radiation image information S according to the following equation:

S=α·S₁+S₂

in step S12.

If the fixed member 11 is a plaster cast, then its radiation absorbing characteristics vary depending on the time that has elapsed from the time when the fixed member 11 was mounted on the subject 12. Therefore, the weighting coefficient α may be set as a function α(t) of the time t that has elapsed from the time when the fixed member 11 was mounted on the subject 12, and the image processor 20 may calculate radiation image information S according to the following equation:

S=α(t)·S₁+S₂

The calculated radiation image information S is displayed on the display device 28 by the display controller 30 in step S13. The display device 28 displays a radiation image of the subject 12 only in the region R1 where the fixed member 11 is mounted on the subject 12.

The radiation image capturing apparatus 10 is not limited to the illustrated embodiment, but may be modified as follows:

Instead of determining the radiation image information S using the radiation image information S₁, S₂ obtained by the two shots, the image processor 20 may determine radiation image information S using radiation image information obtained by three or more shots under different image capturing conditions. If the image processor 20 uses radiation image information S₁, S₂, S₃ obtained by three shots under different image capturing conditions, then the image processor 20 may determine radiation image information S according to the following equation:

S=K₁S₁+K₂·S₂+K₃·S₃+K₄

where K₁, K₂, K₃, K₄ represent coefficients that are determined by the weighting coefficient for extracting the region to be imaged and the gradation characteristics of the image of the region to be imaged. Using coefficients β, γ, the above equation may be simplified into the following equation:

S=β·S₁+γ·S₂+S₃

If the fixed member 11 is a plaster cast, then when it is initially mounted on the subject 12, the fixed member 11 has a temperature higher than the subject 12 because it generates heat. Accordingly, an infrared camera may be used, rather than the CCD camera 36, to acquire subject image information, and the image region identifier 37 may calculate the region R1 of the fixed member 11 and the irradiated region R2 based on the temperature difference.

In the illustrated embodiment, the positions of both the radiation source 14 and the collimator 32 are moved to adjust the irradiated region. However, the collimator 32 may be fixed in position with respect to the radiation source 14 and only the radiation source 14 may be moved to adjust the irradiated region.

Instead of a TFT device, such a device as a CCD (Charge Coupled Device), a CMOS (Complementary Metal Oxide Semiconductor) device or the like may be used for the radiation solid-state detector 18.

The radiation image capturing apparatus may incorporate, instead of the radiation solid-state detector 18 for converting the applied radiation X directly into electric charge information, a radiation detector including a scintillator for converting the applied radiation X into visible light and a detecting device for converting the visible light into electric charge information.

Alternatively, the radiation image capturing apparatus may incorporate a radiation detector of the light readout type for storing the radiation X as an electrostatic latent image and thereafter reading the electrostatic latent image as electric charge information when irradiated with reading light.

Further alternatively, the radiation image capturing apparatus may incorporate a stimulable phosphor panel for storing radiation energy in a phosphor and emitting stimulated light depending on the stored radiation energy when irradiated with stimulating light such as a laser beam or the like.

Although a certain preferred embodiment of the present invention has been shown and described in detail, it should be understood that various changes and modifications may be made therein without departing from the scope of the appended claims.

What is claimed is:

1. A radiation image capturing apparatus for producing radiation image information of a subject by applying a radiation to the subject on which a fixed member is mounted, comprising:
a subject image information acquiring unit for acquiring subject image information of the subject including the fixed member;
an imaging region identifier for processing the acquired subject image information to identify an imaging region of the subject with the fixed member mounted thereon;
an irradiated region controller for controlling an irradiated region to be irradiated with the radiation in order to apply the radiation to the identified imaging region; and
a radiation conversion panel for converting the radiation that has passed through the imaging region into radiation image information.

2. A radiation image capturing apparatus according to claim 1, wherein the subject image information acquiring unit comprises a CCD camera for capturing an image of the subject.

3. A radiation image capturing apparatus according to claim 1, wherein the subject image information acquiring unit comprises an infrared camera for capturing an image of the subject.

4. A radiation image capturing apparatus according to claim 1, wherein the imaging region identifier identifies the imaging region based on inherent image information obtained from the fixed member.

5. A radiation image capturing apparatus according to claim 1, wherein the irradiated region controller comprises irradiated region moving means for moving the irradiated region to the imaging region.

6. A radiation image capturing apparatus according to claim 1, wherein the irradiated region controller comprises an irradiated region adjusting means for adjusting the irradiated region into alignment with the imaging region.

7. A radiation image capturing apparatus according to claim 1, wherein the fixed member comprises a plastic cast or glass fibers mounted on the subject.

8. A radiation image capturing apparatus according to claim 1, further comprising:
an image processor for acquiring radiation image information of the subject from which the fixed member is removed, by processing a plurality of pieces of radiation image information of the imaging region which are captured under different image capturing conditions and acquired from the radiation conversion panel.

9. A method of capturing a radiation image by producing radiation image information of a subject by applying a radiation to the subject on which a fixed member is mounted, comprising the steps of:
acquiring subject image information of the subject including the fixed member;
processing the acquired subject image information to identify an imaging region of the subject with the fixed member mounted thereon;
adjusting an irradiated region to be irradiated with the radiation in order to apply the radiation to the identified imaging region;
irradiating the identified imaging region with the radiation after adjusting the irradiated region; and
converting the radiation that has passed through the imaging region into radiation image information.

10. A method according to claim 9, wherein the imaging region is identified based on inherent image information obtained from the fixed member.

11. A method according to claim 9, wherein the fixed member comprises a plastic cast or glass fibers mounted on the subject.

12. A method according to claim 9, further comprising the step of:
acquiring radiation image information of the subject from which the fixed member is removed, by processing a plurality of pieces of radiation image information of the imaging region which are captured under different image capturing conditions.
